(19) United States
(12) Patent Application Publication
(10) Pub. No.: US 2024/0197260 A1
(43) Pub. Date: Jun. 20, 2024

(54) NOISE REDUCTION IN ANALYTE DATA

(71) Applicant: Dexcom, Inc., San Diego, CA (US)

(72) Inventors: Nunzio CAMERLINGO, Padua (IT); Ilaria SIVIERO, Padua (IT); Martina VETTORETTI, Padua (IT); Simone DEL FAVERO, Padua (IT); Giovanni SPARACINO, Padua (IT); Andrea FACCHINETTI, Padua (IT)

(21) Appl. No.: 18/068,400

(22) Filed: Dec. 19, 2022

(51) Int. Cl. A61B 5/00

(52) U.S. Cl. CPC A61B 5/7203; A61B 5/0004; A61B 5/725; A61B 5/14532

(57) ABSTRACT

Certain aspects of the present disclosure relate to methods and systems for noise reduction in analyte data. Raw analyte data is input into a partitioning algorithm to generate partitioned data. An adaptive filter generates rough filtered partitions reducing a noise component in each partition of the partitioned data. A smoothing algorithm smooths the rough filtered partitions to generate smooth filtered data with reduced noise.

### BACKGROUND

Diabetes is a metabolic condition relating to the production or use of insulin by the body. Insulin is a hormone that allows the body to use glucose for energy, or store glucose as fat.

When a person cats a meal that contains carbohydrates, the food is processed by the digestive system, which produces glucose in the person's blood. Blood glucose can be used for energy or stored as fat. The body normally maintains blood glucose levels in a range that provides sufficient energy to support bodily functions and avoids problems that can arise when glucose levels are too high, or too low. Regulation of blood glucose levels depends on the production and use of insulin, which regulates the movement of blood glucose into cells.

When the body does not produce enough insulin, or when the body is unable to effectively use insulin that is present, blood sugar levels can elevate beyond normal ranges. The state of having a higher than normal blood sugar level is called “hyperglycemia.” Chronic hyperglycemia can lead to a number of health problems, such as cardiovascular disease, cataract and other eye problems, nerve damage (neuropathy), and kidney damage. Hyperglycemia can also lead to acute problems, such as diabetic ketoacidosis-a state in which the body becomes excessively acidic due to the presence of blood glucose and ketones, which are produced when the body cannot use glucose. The state of having lower than normal blood glucose levels is called “hypoglycemia.” Severe hypoglycemia can lead to acute crises that can result in seizures or death.

A diabetes patient can receive insulin to manage blood glucose levels. Insulin can be received, for example, through a manual injection with a needle. Wearable insulin pumps are also potential. Diet and exercise also affect blood glucose levels.

Diabetes conditions are sometimes referred to as “Type 1” and “Type 2.” A Type 1 diabetes patient is typically able to use insulin when it is present, but the body is unable to produce sufficient amounts of insulin, because of a problem with the insulin-producing beta cells of the pancreas. A Type 2 diabetes patient may produce some insulin, but the patient has become “insulin resistant” due to a reduced sensitivity to insulin. The result is that even though insulin is present in the body, the insulin is not sufficiently used by the patient's body to effectively regulate blood sugar levels.

Management of diabetes can present complex challenges for patients, clinicians, and caregivers, as a confluence of many factors can impact a patient's glucose level and glucose trends. To assist patients with better managing this condition, portable or wearable medical devices (e.g., sensors and other types of monitoring and diagnostic devices) as well as a variety of diabetes intervention software applications (hereinafter “applications”) have been developed by various providers.

Because continuous glucose monitoring (CGM) sensors are able to measure glucose concentration in a user, they are important for diabetes management. Analysis of CGM data is beneficial for improving diabetes therapies and, in turn, for improving overall glycemic control for a user. For example, analysis of trends and patterns in CGM data after a meal can help improve insulin dosing by suggesting rescue carbohydrate intake, evaluating glucose variability, quantifying the effectiveness of a therapy, and improving visualization of relevant CGM patterns.

### BRIEF DESCRIPTION OF THE DRAWINGS

So that the manner in which the above-recited features of the present disclosure can be understood in detail, a more particular description, briefly summarized above, may be had by reference to aspects, some of which are illustrated in the drawings. It is to be noted, however, that the appended drawings illustrate only certain typical aspects of this disclosure and are therefore not to be considered limiting of its scope, for the description may admit to other equally effective aspects.

FIG. 1 is a diagram conceptually illustrating an example continuous analyte monitoring system including example continuous analyte sensor(s) with sensor electronics, in accordance with certain aspects of the present disclosure.

FIG. 2 illustrates an example of a method of performing noise reduction on raw analyte data, according to some embodiments disclosed herein.

FIG. 3 is an example flow diagram illustrating a process for reducing noise in analyte data that may be used in connection with implementing embodiments of the present disclosure.

FIG. 4 is a graph of example raw analyte data, according to some embodiments disclosed herein.

FIG. 5 illustrates an example of a partition of the raw analyte data of FIG. 4, according to some embodiments disclosed herein.

FIG. 6 illustrates an example of a series of partitions of the raw analyte data of FIG. 4, according to certain embodiments of the present disclosure.

FIG. 7 illustrates an example of rough filtered partitions in the partitions of FIG. 5, according to certain embodiments of the present disclosure.

FIG. 8 illustrates an example of rough filtered partitions in the series of partitions of FIG. 6, according to certain embodiments of the present disclosure.

FIG. 9 illustrates an example of smooth filtered data with a reduced noise component, according to some embodiments disclosed herein.

FIG. 10 illustrates an example graph of analyte data with a missing portion, according to certain embodiments disclosed herein.

FIG. 11 illustrates an example of data mirroring at an end of raw analyte data, according to certain embodiments disclosed herein.

FIG. 12 illustrates an example of smooth filtered data for the end of raw analyte data of FIG. 1, according to certain embodiments disclosed herein.

FIG. 13 illustrates an example of the smooth filtered data of FIG. 12 with mirrored data removed, according to certain embodiments disclosed herein.

To facilitate understanding, identical reference numerals have been used, where possible, to designate identical elements that are common to the figures. It is contemplated that elements disclosed in one aspect may be beneficially utilized on other aspects without specific recitation.

### DETAILED DESCRIPTION

Despite the constant improvements made to analyte sensors in terms of accuracy, analyte measurements continue to be affected by measurement noise that impacts the true signal.

Reducing the sensor's error increases accuracy of analyte data which improves the reliability of the decisions made by users and caregivers based on the analyte data. To this end, numerous filtering/smoothing techniques have been proposed to enhance the quality of the signal and to reduce the noise component of the signal, thus improving the signal-to-noise (SNR) ratio of analyte data.

For example, digital low-pass filters have been used to reduce analyte measurement noise. For instance, for an existing data set, zero-phase Butterworth filters may be used, which process data in both forward and backward directions, introducing no delay. However, once the parameters have been fixed (e.g., the cutoff frequency), these filters are no longer able to adapt their “aggressiveness” to cope with the SNR variability of the analyte signal. Therefore, digital low-pass filters are generally suboptimal. Other smoothing/filtering approaches have been attempted but fail to provide sufficient handling of measurement noise.

Accordingly, the embodiments described herein provide systems and methods of reducing noise in analyte data. In particular, the embodiments herein provide a health management system, including a display device and an analyte monitoring system, including an analyte sensor (c.g., CGM sensor) configured to generate analyte measurements (e.g., glucose measurements) for transmission to the display device. The display device includes a processor configured to execute a software application for receiving and processing the analyte data (e.g., CGM data) indicative of the analyte measurements generated by the analyte sensor. The software application may use a noise reduction algorithm to reduce the noise associated with the received analyte data. The software application may alternatively be configured to send the received analyte data to a server that executes the noise reduction algorithm for reducing the noise in the analyte data.

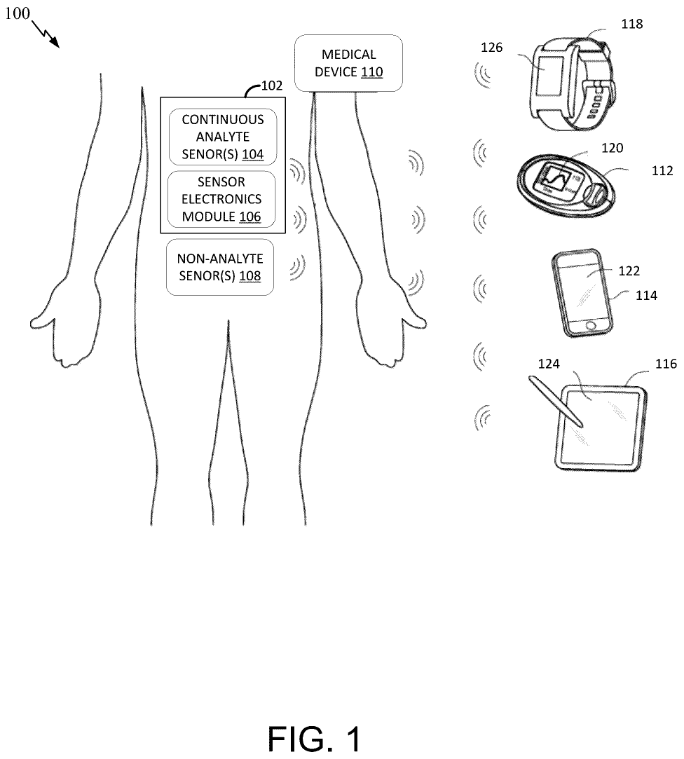

Noise in the analyte data demonstrates a temporal correlation or a relationship between the noise and time, in that the noise randomly changes over time, assuming values that are statistically linked to the previous values. To identify the noise component in the analyte data, the noise reduction algorithm partitions the analyte data (i.c., signal trace) into consecutive partitions cach with a length L (L refers to a length of a window of time, e.g., 1 minute, 5 minutes, 30 minutes, 1 hour, etc.). Each partition corresponds to a portion of the analyte data received over the corresponding time period with length L. For each partition, the noise reduction algorithm stochastically estimates the filtered signal and the noise variance. The noise reduction algorithm is configured to then join and smooth the filtered signals from each partition to generate a single refined signal with improved accuracy through noise reduction. Note that, the analyte data herein refers to a stream of analyte measurements received by the display device over a period of time from the analyte monitoring system. For example, the analyte data may include analyte measurements associated with the past 30 minutes or another period of time. Examples of the analyte monitoring system and the display device are described in more detail in relation to FIG. 1.

FIG. 1 illustrates an analyte monitoring system 100 including an example continuous analyte sensor system 102, non-analyte sensor(s) 108, medical device 110, and a plurality of display devices 112, 114, 116, and 118, in accordance with certain aspects of the present disclosure. The components of the analyte monitoring system 100 are configured to operate continuously to monitor one or more analytes of a user, in accordance with certain aspects of the present disclosure.

Continuous analyte monitoring system 102, in the illustrated embodiment, includes sensor electronics module 106 and one or more continuous analyte sensor(s) 104 (individually referred to herein as continuous analyte sensor 104 and collectively referred to herein as continuous analyte sensors 104) associated with sensor electronics module 106. Sensor electronics module 106 may be in wireless communication (e.g., directly or indirectly) with one or more of display devices 112, 114, 116, and 118. In certain embodiments, sensor electronics module 106 may also be in wireless communication (c.g., directly or indirectly) with one or more medical devices, such as medical devices 110 (individually referred to herein as medical device 110 and collectively referred to herein as medical devices 110), and/or one or more other non-analyte sensors 108 (individually referred to herein as non-analyte sensor 108 and collectively referred to herein as non-analyte sensor 108).

In certain embodiments, a continuous analyte sensor 104 may comprise a sensor for detecting and/or measuring analyte(s). The continuous analyte sensor 104 may be a multi-analyte sensor configured to continuously measure two or more analytes or a single analyte sensor configured to continuously measure a single analyte as a non-invasive device, a subcutaneous device, a transcutaneous device, a transdermal device, and/or an intravascular device. In certain embodiments, the continuous analyte sensor 104 may be configured to continuously measure analyte levels of a user using one or more measurement techniques, such as enzymatic, chemical, physical, electrochemical, spectrophotometric, polarimetric, calorimetric, iontophoretic, radiometric, immunochemical, and the like. In certain aspects the continuous analyte sensor 104 provides a data stream indicative of the concentration of one or more analytes in the user. The data stream may include raw data signals, which may then be converted into a calibrated and/or filtered data stream used to provide estimated analyte value(s) to the user.

In certain embodiments, continuous analyte sensor 104 may be a multi-analyte sensor, configured to continuously measure multiple analytes in a user's body. For example, in certain embodiments, the continuous multi-analyte sensor 104 may be a single multi-analyte sensor configured to measure two or more of glucose, insulin, lactate, ketones, pyruvate, and potassium in the user's body.

In certain embodiments, the continuous analyte sensor 104 may be a continuous glucose monitor (CGM). Some examples of a continuous glucose monitor include a glucose monitoring sensor. In some embodiments, glucose monitoring sensor is an implantable sensor, such as described with reference to U.S. Pat. No. 6,001,067 and U.S. Patent Publication No. US-2011-0027127-A1. In some embodiments, the glucose monitoring sensor is a transcutaneous sensor, such as described with reference to U.S. Patent Publication No. US-2006-0020187-A1. In yet other embodiments, the glucose monitoring sensor is a dual electrode analyte sensor, such as described with reference to U.S. Patent Publication No. US-2009-0137887-A1. In still other embodiments, the glucose monitoring sensor is configured to be implanted in a host vessel or extracorporeally, such as the sensor described in U.S. Patent Publication No. US-2007-0027385-A1. These patents and publications are incorporated herein by reference in their entirety.

As used herein, the term “continuous” may mean fully continuous, semi-continuous, periodic, etc. Such continuous monitoring of analytes is advantageous in diagnosing and staging a disease given the continuous measurements provide continuously up to date measurements as well as information on the trend and rate of analyte change over a continuous period. Such information may be used to make more informed decisions in the assessment of glucose homeostasis and treatment of diabetes.

In certain embodiments, sensor electronics module 106 includes electronic circuitry associated with measuring and processing the continuous analyte data, including prospective algorithms associated with processing and calibration of the analyte data. Sensor electronics module 106 can be physically connected to continuous analyte sensor(s) 104 and can be integral with (non-releasably attached to) or releasably attachable to continuous analyte sensor(s) 104. Sensor electronics module 106 may include hardware, firmware, and/or software that enables measurement of levels of analyte(s) via a continuous analyte sensor(s) 104. For example, sensor electronics module 106 can include a potentiostat, a power source for providing power to the sensor, other components useful for signal processing and data storage, and a telemetry module for transmitting data from the sensor electronics module to one or more display devices. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, and/or a processor.

Display devices 112, 114, 116, and/or 118 are configured for displaying displayable analyte data, including analyte data, which may be transmitted by sensor electronics module 106. Each of display devices 112, 114, 116, or 118 can include a display such as a touchscreen display 120, 122, 124, or 126 for displaying analyte data to a user and/or receiving inputs from the user. For example, a graphical user interface (GUI) may be presented to the user for such purposes. In some embodiments, the display devices may include other types of user interfaces such as a voice user interface instead of, or in addition to, a touchscreen display for communicating analyte data to the user of the display device and/or receiving user inputs.

In some embodiments, one, some, or all of the display devices include a processor to perform a noise reduction algorithm on the analyte data communicated from the sensor electronic module (e.g., in one or more data packages transmitted to respective display devices). In some embodiments, one or more of the display devices may execute the noise reduction algorithm to generate smooth filtered data and provide the smooth filtered data to another of the display devices to display, without any additional prospective processing required, for display of the analyte data.

The plurality of display devices may include a custom display device especially designed for displaying certain types of displayable analyte data received from sensor electronics module. In certain embodiments, the plurality of display devices may be configured for providing alerts/alarms based on the displayable analyte data. Display device 112 is an example of such a custom device. In some embodiments, one of the plurality of display devices is a smartphone, such as display device 114 which represents a mobile phone, using a commercially available operating system (OS), and configured to display a graphical representation of the continuous sensor data (c.g., including current and historic data). Other display devices can include other hand-held devices, such as display device 116 which represents a tablet, display device 118 which represents a smart watch, medical device 110 (e.g., an insulin delivery device or a blood glucose meter), and/or a desktop or laptop computer (not shown).

As mentioned, sensor electronics module 106 may be in communication with a medical device 110. Medical device 110 may be a passive device in some example embodiments of the disclosure. For example, medical device 110 may be an insulin pump for administering insulin to a user. For a variety of reasons, it may be desirable for such an insulin pump to receive and track analyte data (c.g., glucose, potassium, lactate, insulin, ketone, and/or pyruvate values) transmitted from continuous analyte monitoring system 102.

The term “analyte” as used herein is a broad term used in its ordinary sense, including, without limitation, to refer to a substance or chemical constituent in a biological fluid (for example, blood, interstitial fluid, cerebral spinal fluid, lymph fluid or urine) that can be analyzed. In the examples described below, the analyte is glucose in the blood stream of the user. However, the concentration of any analyte or any time-varying value that can be measured may be predicted using the approach described herein. For example, analytes can include naturally occurring substances, artificial substances, metabolites, and/or reaction products. Analytes for measurement by the devices and methods may include, but may not be limited to, potassium, glucose, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); androstenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, glucose-6-phosphate dehydrogenase, hemoglobin A, hemoglobin S, hemoglobin C, hemoglobin D, hemoglobin E, hemoglobin F, D-Punjab, hepatitis B virus, HCMV, HIV-1, HTLV-1, MCAD, RNA, PKU, Plasmodium vivax, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sisomicin; somatomedin C; specific antibodies recognizing any one or more of the following that may include (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniac, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain implementations. Ions are a charged atom or compounds that may include the following (sodium, potassium, calcium, chloride, nitrogen, or bicarbonate, for example). The analyte can be naturally present in the biological fluid, for example, a metabolic product, a hormone, an antigen, an antibody, an ion and the like. Alternatively, the analyte can be introduced into the body or exogenous, for example, a contrast agent for imaging, a radioisotope, a chemical agent, a fluorocarbon-based synthetic blood, a challenge agent analyte (e.g., introduced for the purpose of measuring the increase and or decrease in rate of change in concentration of the challenge agent analyte or other analytes in response to the introduced challenge agent analyte), or a drug or pharmaceutical composition, including but not limited to exogenous insulin; glucagon, ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA), and intermediaries in the Citric Acid Cycle.

As described above, continuous analyte monitoring system 102 is configured to continuously measure one or more analytes and transmit the resulting analyte measurements, in the form of analyte data, to a display device (c.g., display device 112, 114, 116, and/or 118), which is configured with a noise reduction algorithm to reduce signal noise associated with the analyte data before the analyte data is displayed to the user and/or analyzed for generating decision support recommendations. In certain embodiments, instead of a display device, the noise reduction algorithm may be executed on a server in data communication with the display device and/or continuous analyte monitoring system 102. In such embodiments, the server uses the noise reduction algorithm to reduce signal noise associated with the analyte data and transmits the filtered and smoothed analyte data to the display device. In some embodiments, algorithms described herein may be implemented wholly or in-part on the display device, a server, and/or another device in communication with the display device and/or server.

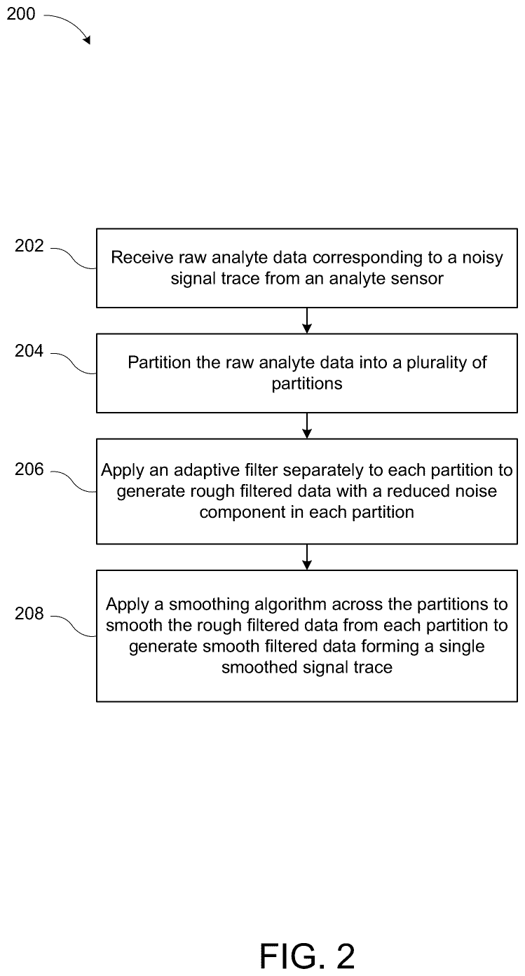

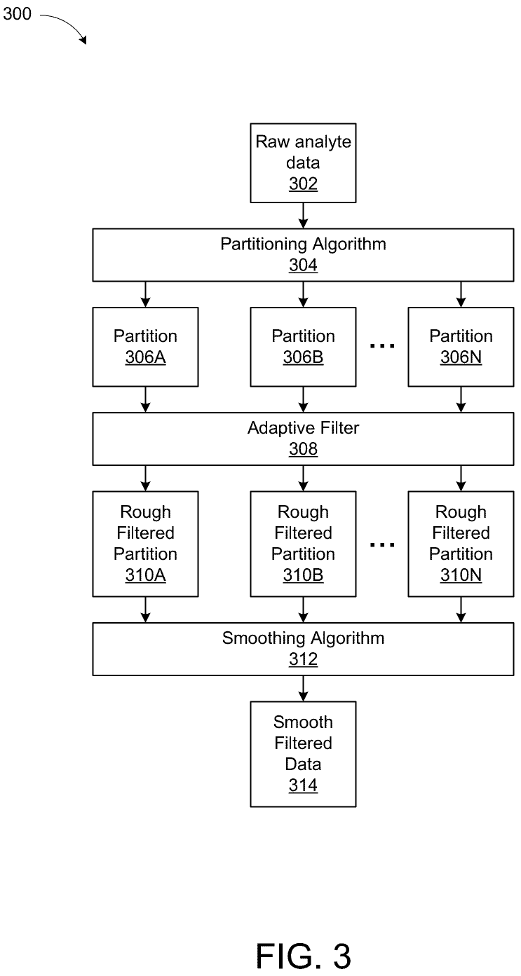

FIG. 2 illustrates an example of a method 200 of performing noise reduction on raw analyte data, according to some embodiments disclosed herein. Method 200 may be wholly or in-part performed by a computing device, such as display device (e.g., display device 118-116) and/or a server and/or another device in communication with one or both of the display device and/or the server. Method 200 is described below by reference to FIGS. 3-13. In particular, FIG. 3 provides a graphical representation of the raw analyte data and the various algorithms used to reduce noise in the raw analyte data.

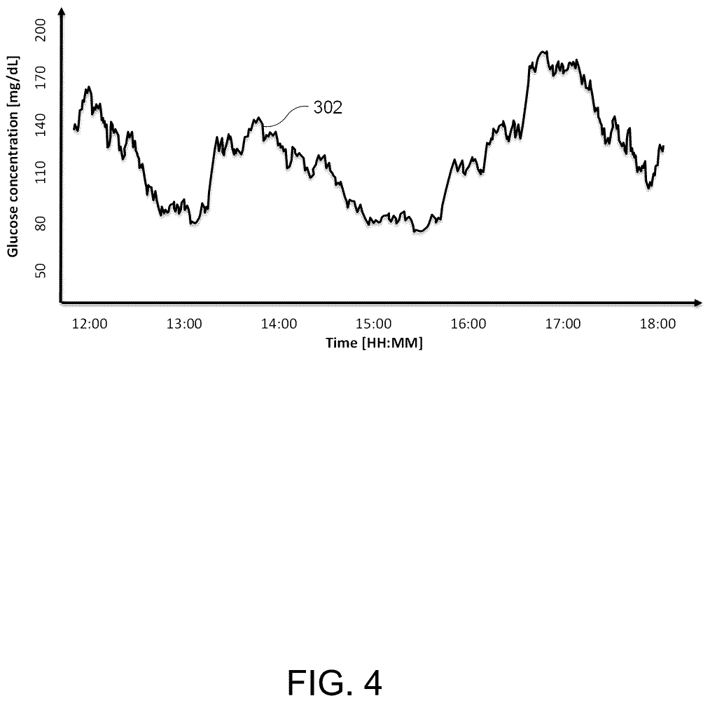

In the illustrated embodiment, the method 200 includes, at block 202, receiving raw analyte data corresponding to a noisy signal trace from an analyte sensor, such as continuous analyte sensor 104. FIG. 3 illustrates the raw analyte data as raw analyte data 302. FIG. 4 illustrates an example graph or signal trace of raw analyte data 302, according to some embodiments disclosed herein. Raw analyte data 302 represents data that is generated over time and forms a signal trace. Raw analyte data 302 may be provided to the computing device by continuous analyte monitoring system 102.

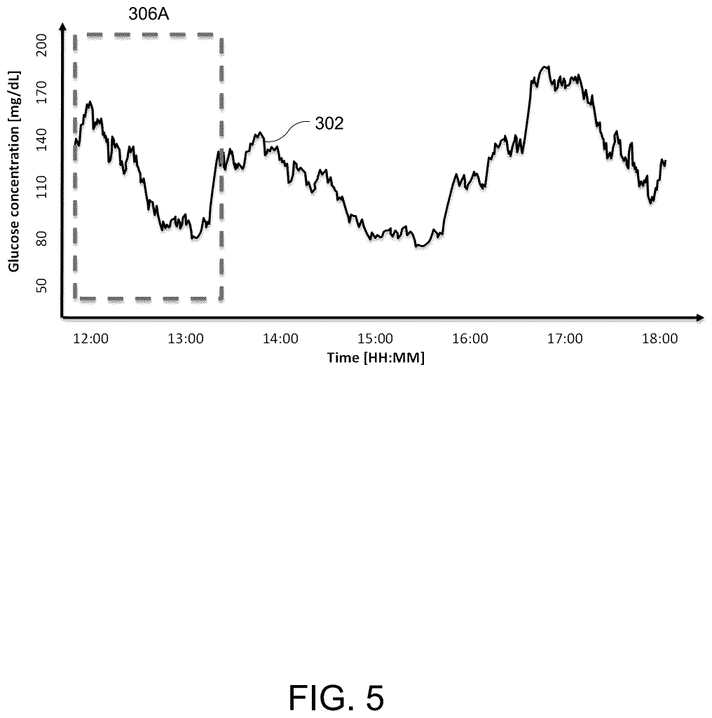

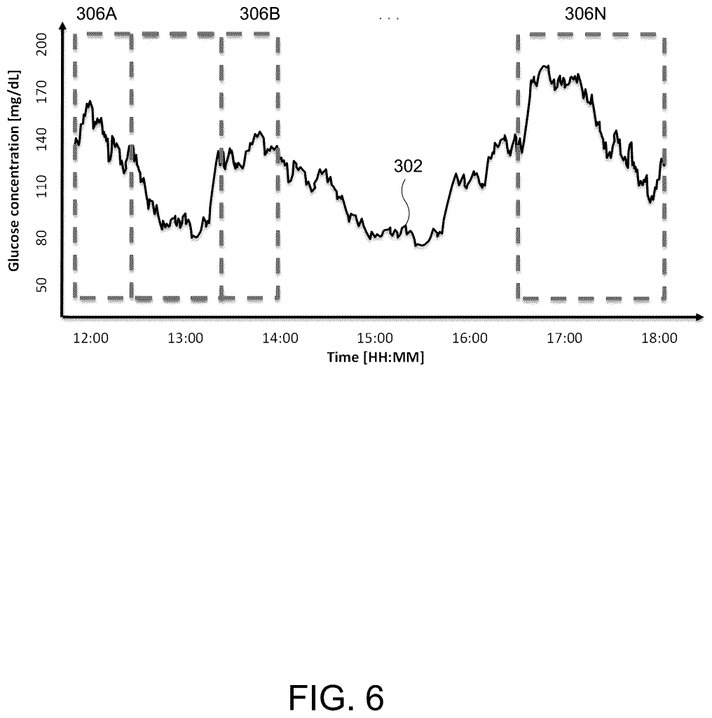

At block 204, method 200 includes partitioning raw analyte data 302 into a plurality of partitions. For example, partitioning algorithm 304 of FIG. 3 is shown as receiving raw analyte data 302 and is configured to separate raw analyte data 302 into partitions 306A-N. FIGS. 5 and 6 illustrate partitions 306A-N as applied to the raw analyte data 302.

In particular, to reduce a time-varying noise component in raw analyte data 302, first, the computing device utilizes partitioning algorithm 304 to partition raw analyte data 302 partitions 306A through 306N, where N (any positive integer) indicates that raw analyte data 302 can be partitioned into any number of partitions. The partitioning is performed so that the variance in the noise component can be assessed on an individual level within each of the partitions 306A-N. In some embodiments, the size of each of the partitions 306A-N is uniform. In other embodiments, the size of at least one of partitions 306A-N may vary relative to one or more of the other partitions 306A-N. In some embodiments, partitions 306A-N are equally-spaced and centered at 2 [symbol] +1, where [symbol] is a free hyperparameter corresponding to the time duration of each partition. In testing, a sensitivity analysis was used to analyze different values of & ranging in number of samples. The analysis applied a root mean square estimation to a test analyte dataset and indicated that a value of [symbol] =20 analyte samples yielded a better fit over the test analyte dataset. Different values of [symbol] may also be used.

In some embodiments, partitions 306A-N are separate from one another, meaning that they are adjoining but not overlapping one another. In other embodiments, partitions 306A-N each overlap one or more adjacent partitions. An example of overlapping partitions 306A-N is provided in FIG. 6. In some embodiments, the overlap of adjacent partitions is a 2 [symbol] point overlap, as illustrated and described further with reference to FIG. 6 below.

At block 206, the computing device applies an adaptive filter separately to each of partition 306A-N to generate rough filtered partitions 310A-N. FIG. 3 provides a graphical representation of an adaptive filter 308 and rough filtered partitions 310A-N.

For example, once raw analyte data 302 is partitioned into partitions 306A-N, adaptive filter 308 is applied by the computing device to each of the partitions separately (i.e., individually). Adaptive filter 308 is applied to each of the partitions 306A-306N separately because the noise component in each of the partitions 306A-306N may be different from a noise component of another of partitions 306A-N. By addressing each of partitions 306A-306N separately, a filtering aggressiveness may be determined which more closely matches the corresponding noise component of each of partitions 306A-306N.

In some embodiments, the analyte data from each partition 306A-N can be modelled as a random process y(k), where y(1), y(2), . . . , y(k), . . . , y(n) are samples of a realization of the process collected in equally-spaced time instants t=kTs, k=1,2, . . . , n, and T_{S} is the sampling period of the analyte sensor (e.g., continuous analyte sensor 104). Each sampling time k, y(k) may be given by the sum of two contributions:

$$y ( k ) = u ( k ) + w ( k ) \qquad ( 1 )$$

Here, u(k) is the true analyte component of the analyte data and w(t) is the noise component affecting the analyte measurement (the noise component is assumed to be additive).

The vectors y=[y(1), y(2), . . . , y(n)]^{T}, u=[u(1), u(2), . . . , u(n)]^{T}, and w=[w(1), w(2), . . . , w(n)]^{T} may contain the samples of the realization of the true analyte component and the noise component, respectively, collected at time instants k=1,2, . . . , n.

To estimate u(k) from raw analyte data 302, defined as y(k), it is possible to compute the estimate û, linearly dependent on y, that minimizes E [||u−û||²]. Specifically, if u and w are uncorrelated zero-mean random vectors with a priori covariance matrices denoted by Σᵤ and Σ_{w}, respectively, then the linear mean square estimate of u given y is the solution of the problem:

$$arg {min}_{u} \{ {( y - u )}^{T} {Σ}_{w}^{- 1} ( y - u ) + {u}^{T} {Σ}_{u}^{- 1} u \} \qquad ( 2 )$$

that is:

$$\hat{u} = {( {Σ}_{w}^{- 1} + {Σ}_{u}^{- 1} )}^{- 1} {Σ}_{w}^{- 1} y \qquad ( 3 )$$

In some embodiments, adaptive filter 308 defines a statistical prior knowledge on the unknown true analyte component (u(k) and the unknown noise component w(k) in Eq. (1)). An a priori model may be used to describe analyte data of reduced noise on a uniformly spaced discrete grid. Here, a multiple-integrated white noise model may be used as an a priori model. Using a multiple-integrated white noise model is particularly advantageous because the only unknown parameter here is the variance of the noise component of the multiple-integrated white noise model, which can be estimated from partitions 306A-N.

Although the number of integrators, m, for the multiple-integrated white noise model cannot be analytically determined, in testing, a multiple-integrated white noise model of cascade of m=2 integrators (the so-called integrated random walk):

$$u ( k ) = 2 u ( k - 1 ) - u ( k - 2 ) + v ( k ) \qquad ( 4 )$$

proved to represent a satisfactory a priori model, where v(k) is a zero-mean Gaussian noise with unknown variance equal to λ². From Eq. (4), Eu can be obtained as:

$${Σ}_{u} = {{λ}^{2} ( {F}^{T} F )}^{- 1}$$

where F is the square n-dimensional lower-triangular Toeplitz matrix, whose first column is [1, −2, 1, 0, . . . , 0]^{T}.

To describe the measurement noise affecting u(k), i.e., w(k) of Eq. (1), given that the noise component of partitions 306A-N are correlated, an AR(2) driven by a stationary white noise ϵ₁(k), with a zero-mean and an unknown variance equal to σ²:

$${w}_{1} ( k ) = {a}_{1} {w}_{1} ( k - 1 ) + {a}_{2} {w}_{1} ( k - 2 ) + {ϵ}_{1} ( k ) \qquad ( 5 )$$

where: ϵ₁(k)=WN(0, σ²)

The values of a₁, and a₂ may be fixed to the median values. Embodiments of a₁, and a₂ may be equal to 1.30, and −0.42, respectively. Because, as described above, the noise component in each of partitions 306A-N is not uniform, each of partitions 306A-N is analyzed separately. From Eq. (5), Σ_{w} can be obtained as:

$${Σ}_{w} = {{σ}^{2} ( {A}^{T} A )}^{- 1}$$

where A is the square n-dimensional lower-triangular Toeplitz matrix, whose first column is [1, a₁, a₂, 0, . . . , 0]^{T}. The time-correlation among the noise component of the analyte data is taken into account by Σ_{w} which, unlike a traditional assumption of white measurement noise, has non-null elements also outside the principal diagonal. Once defined, Σᵤ and Σ_{w}, Eq. (3) turns into:

$$\hat{u} = {( {A}^{T} A + γ {F}^{T} F )}^{- 1} {A}^{T} A y \qquad ( 6 )$$

where

$$γ = \frac{{σ}^{2}}{{λ}^{2}}$$

is a regularization parameter that weights the fidelity to the data (first term in the cost function of Eq. (2)) and the roughness of the estimate (second term in the cost function of Eq. (2)). Here, large values of γ may lead to oversmoothing, i.e., an estimate û poorly influenced by the measurement y, while small values of γ may lead to undersmoothing, i.e., an estimate û that accurately fits the measurements y, poorly attenuating the noise.

As shown above, because both σ² and λ² are unknown, the value of γ is also unknown. Furthermore, given the inter-subject variability of the SNR, the value of γ cannot be set using a population-based value, but should instead be personalized. To address the intra-subject variability of the SNR, the value of γ may be derived separately for each of partitions 306A-N. For each partition, the value of γ may also be adaptively tuned. To do so, the problem of Eq. (6) may be iteratively solved for several trial values of γ, until the following condition is satisfied:

$$\frac{WRSS ( γ )}{n - q ( γ )} = γ \frac{WESS ( γ )}{q ( γ )} \qquad ( 7 )$$

where WRSS(γ)=(y−û)^{T}A^{T}A(y−û) is the weighted residual sum of squares, WESS(γ)=û^{T}F^{T}Fû is the weighted estimates sum of squares, and q(γ)=trace[A^{T}(A^{T}A+γF^{T}F)⁻¹A)] is the degree of freedom of the estimator.

As γ is determined, the estimate of the measurement noise variance σ² is given by:

$${\hat{σ}}^{2} = \frac{WRSS ( γ )}{n - q ( γ )} \qquad ( 8 )$$

and the estimate \hat{λ}² is obtained by dividing Eq. (8) by γ.

In this framework, it is also possible to compute the covariance of the estimation error ũ=u−û as:

$$cov ( \tilde{u} ) = {{σ}^{2} ( {A}^{T} A + γ {F}^{T} F )}^{- 1}$$

whose square root diagonal elements could be used to obtain the confidence interval (CI) of the estimate û.

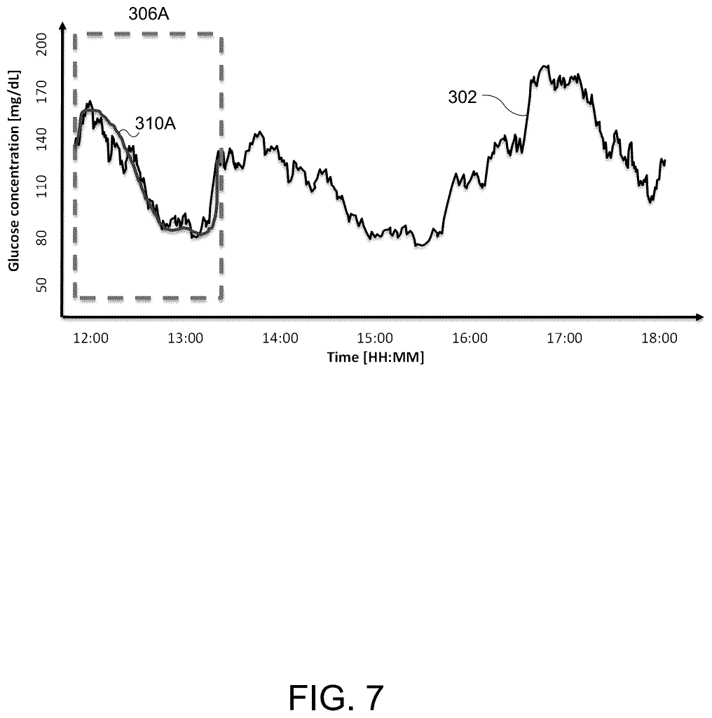

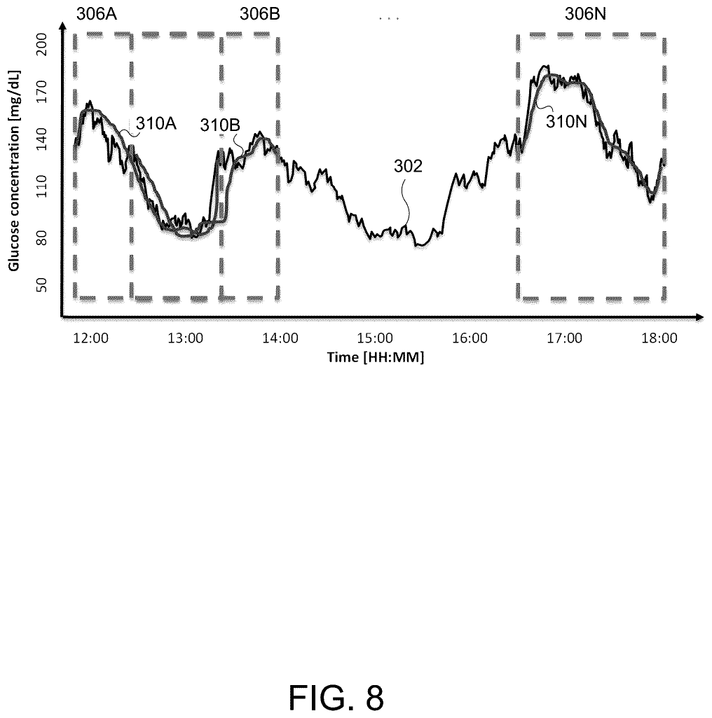

As described above, adaptive filter 308 is implemented for each of partitions 306A-N. Adaptive filter 308 estimates û in Eq. (6). As such, adaptive filter 308 generates rough filtered partitions 310A-N corresponding to each of partitions 306A-N with the noise component of each of partitions 306A-N independently reduced. In other words, adaptive filter 308 determines and applies a corresponding filter aggressiveness for each partition 306A-N and calculates a noise variance for each partition as well. Examples of the resulting rough filtered partitions 310A-N are illustrated in FIGS. 7 and 8.

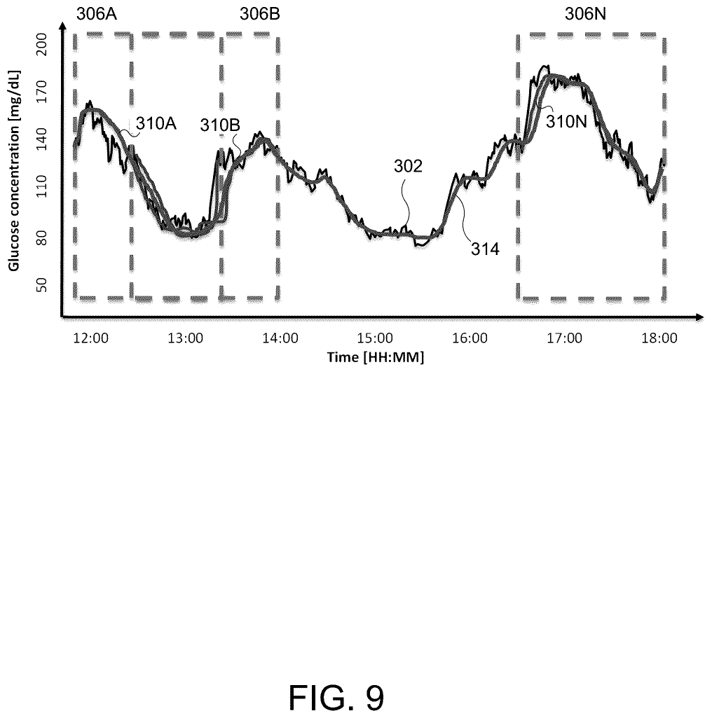

At block 208, the computing device applies a smoothing algorithm across the rough filtered partitions 310A-N to smooth the rough filtered partitions 310A-N and generate smooth filtered data forming a single smoothed signal trace, as described below in more detail. A graphical representation of the smoothing algorithm is provided as smoothing algorithm 312 in FIG. 3 which is configured to generate smooth filtered data 314. An example of smooth filtered data 314 is shown in FIG. 9.

Rough filtered partitions 310A-N have reduced noise components in relation to partitions 306A-N but, in reducing the noise component individually for each partition, the overall signal trace from the original raw analyte data 302 may become disjointed and discontinuous at partition thresholds or other locations within the signal trace. To reassemble the signal trace, rough filtered partitions 310A-N are provided as inputs to a smoothing algorithm 312. Smoothing algorithm 312 may be configured to reconstruct a signal with reduced noise and continuity throughout. In some embodiments, smoothing algorithm 312 executes a kernel smoother. The kernel smoother may effectively reduce or eliminate jumps or discontinuities around the boundaries of neighboring partitions to stitch rough filtered partitions 310A-N back together.

In some embodiments a noise variance profile may be generated by collecting the noise variance from each rough filtered partition 310A-N. For example, let us denote each of rough filtered partition 310A-N as the signal estimate from data of each partition i, of each of partition 306A-N, as û⁽ⁱ⁾(k), and the noise variance as \hat{σ}²^{⁽¹⁾}(k), k=1, 2, . . . , 2 [symbol] +1. For example, û⁽¹⁾(k) is obtained by smoothing y(k), k=1,2, . . . , 2 [symbol] +1, while û⁽²⁾(k) is obtained by smoothing y(k), k=2,3, . . . , 2 [symbol] +2.

In some embodiments, the smoothing algorithm 312 may identify a kernel K having the same width of partitions 306A-N, for example, 2 [symbol] +1 equally-spaced 5-min points. Smoothing algorithm 312 outputs smooth filtered data 314. In particular, rough filtered partitions 310A-N at time c=2 [symbol] +1, 2 [symbol] +2, . . . , n−2 [symbol] are given by the weighted average of rough filtered partitions 310A-N in the neighboring windows:

$$\hat{u} ( c ) = \frac{{Σ}_{i = 1}^{2 ℓ + 1} {\hat{u}}^{( c - i + 1 )} ( i ) ( 2 ℓ + 2 - i )}{{Σ}_{i = 1}^{2 ℓ + 1} ( i )}$$

For example, the first point of the smoothed signal û(2 [symbol] +1) is obtained by weighting the points û⁽¹⁾(2 [symbol] +1), û²(2 [symbol] ), . . . , [symbol] (1).

In some embodiments, a continuous profile of \hat{σ}² at time c=2 [symbol] +1,2 [symbol] +2, . . . , n−2 [symbol] , may be constructed. The continuous profile may allow for tracking of intraindividual noise variability:

$${\hat{σ}}^{2} ( c ) = \frac{{Σ}_{i = 1}^{2 ℓ + 1} {\hat{σ}}^{{2}^{( c - i + 1 )}} ( i ) ( 2 ℓ + 2 - i )}{{Σ}_{i = 1}^{2 ℓ + 1} ( i )}$$

In other words, the continuous profile may describe how the noise component of raw analyte data 302 varies with time.

In some embodiments, each of rough filtered partitions 310A-N in partitions centered at point c may be weighted more than rough filtered partitions 310A-N in neighboring partitions. Thus, [symbol] may be selected as a Gaussian kernel centered in c=2 [symbol] +1,2 [symbol] +2, . . . , n−2 [symbol] , with standard deviation Λ:

$$= ( c , Λ )$$

Standard deviation Λ may be a second free hyperparameter which defines the standard deviation of the kernel K. To find suitable values, different values of [symbol] were tested ranging in quantity of samples in the analyte data, and different values of Λ ranging in quantity of samples in the analyte data. The values providing the lowest median RMSE over the test analyte dataset were [symbol] =20 points of analyte data, and Λ=10 points of analyte data.

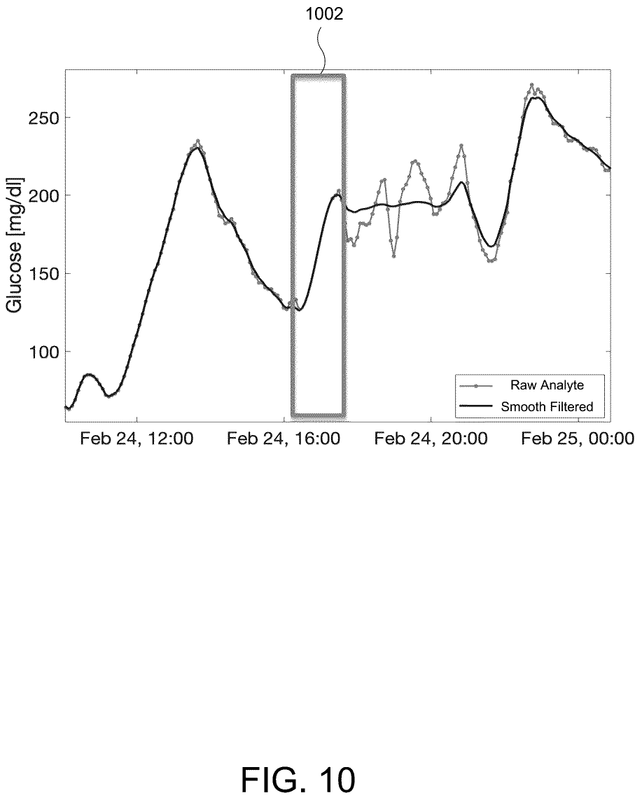

FIG. 10 illustrates an example of addressing missing data 1002 of the raw analyte data, according to certain embodiments disclosed herein. As described above, using smoothing algorithm 312, the computing device is configured to smooth discontinuities in raw analyte data 302. For example, if raw analyte data 302 has portions of missing data 1002, using algorithm 300, the computing device may be configured to extrapolate smooth filtered data 314 to correspond to missing data 1002 and remedy the discontinuity based on surrounding portions of raw analyte data 302.

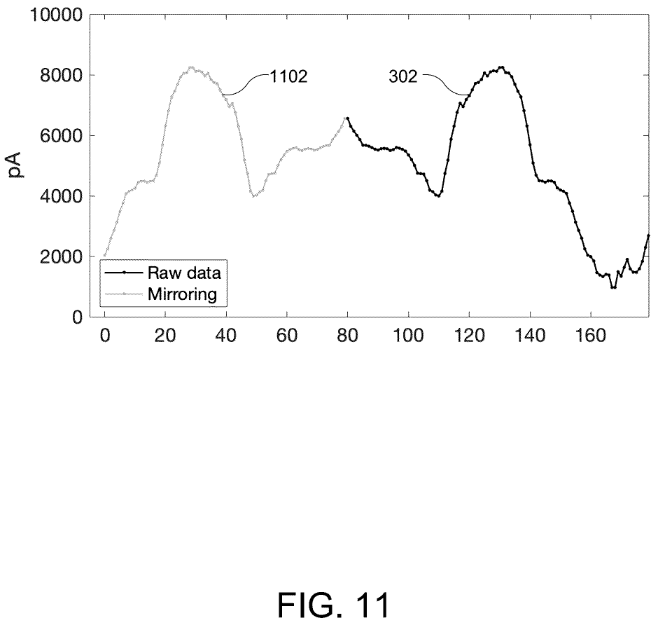

In some embodiments, the execution of a kernel smoother by smoothing algorithm 312 may not properly consider the final estimates û and \hat{σ} in the beginning and the final portions of raw analyte data 302 of duration 2 [symbol] . In some embodiments, algorithm 300 may implement a “data mirroring”. To use data mirroring, the first 2 [symbol] portions of raw analyte 302 in y are duplicated, flipped and arranged before the first point y(1) of raw analyte data 302. An example of data mirroring is shown in FIG. 11. Similarly, the last 2 [symbol] portions of raw analyte data 302 in y are duplicated, flipped and put after the last point y(n) of the raw analyte data 302. By data mirroring at the beginning and final portion of raw analyte data 302, a sufficient amount of data points (i.e., 2 [symbol] +1 samples), statistically similar to the actual raw analyte data 302, are available as input to the kernel smoother of smoothing algorithm 312 at the beginning/final portions of the raw analyte data 302.

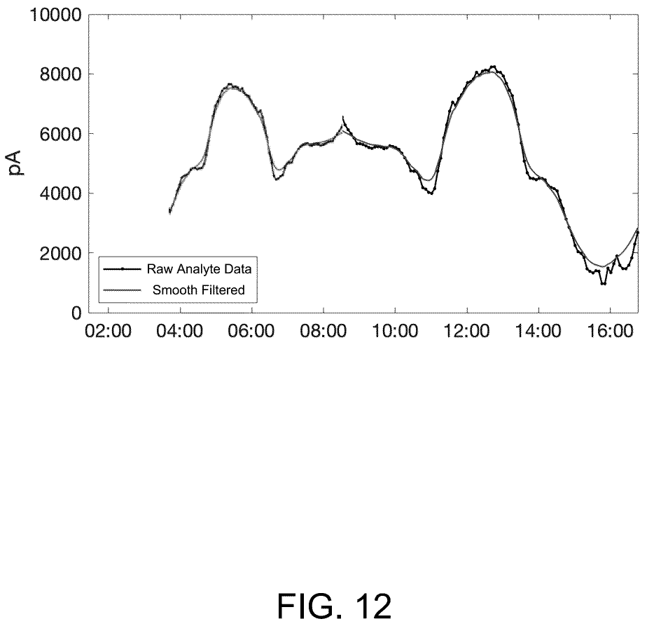

FIG. 12 illustrates an example of smooth filtered data 314 for the end of raw analyte data 302 of FIG. 1, according to certain embodiments disclosed herein. With mirrored data 1102 in place, raw analyte data 302 may, as described above, be partitioned by the partitioning algorithm 304, filtered by the adaptive filter 308, and smoothed by the smoothing algorithm 312 to form smooth filtered data 314.

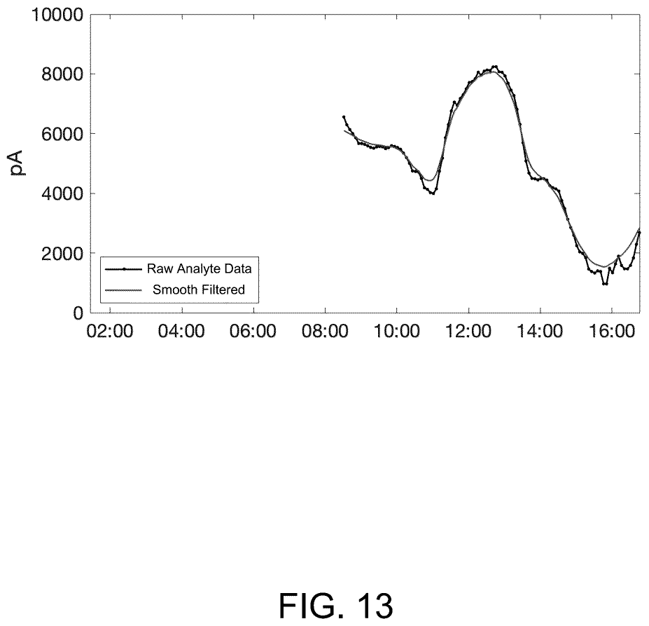

FIG. 13 illustrates an example of the smooth filtered data 314 of FIG. 12 with mirrored data 1102 removed, according to certain embodiments disclosed herein. In the illustrated embodiment, mirrored data 1102 is removed and smooth filtered data is shown with respect to raw analyte data 302.

### Additional Considerations

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims.

As used herein, a phrase referring to “at least one of” a list of items refers to any combination of those items, including single members. As an example, “at least one of: a, b, or c” is intended to cover a, b, c, a-b, a-c. b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112(f) unless the element is expressly recited using the phrase “means for” or, in the case of a method claim, the element is recited using the phrase “step for.”

While various examples of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described example examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular example of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The term “comprising as used herein is synonymous with “including,” “containing,” or “characterized by” and is inclusive or open-ended and does not exclude additional, unrecited elements or method steps.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, cach numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific examples and examples described herein, but rather to also cover all modification and alternatives coming with the true scope and spirit of the invention.

What is claimed is:

1. A method for noise reduction in analyte data, the method comprising:
receiving, by a processor, raw analyte data corresponding to a noisy signal trace from an analyte sensor;
partitioning, by the processor, the raw analyte data into a plurality of partitions;
applying, by the processor, an adaptive filter separately to each partition to generate rough filtered partitions with a reduced noise component in each partition; and
applying, by the processor, a smoothing algorithm across the partitions to smooth the rough filtered partitions from each partition to generate smooth filtered data forming a single smoothed signal trace.

2. The method of claim 1, further comprising determining, by the processor, a noise variance profile for the raw analyte data based on a noise component determined for each of the plurality of partitions.

3. The method of claim 1, wherein the adaptive filter is configured to implement a filtering aggressiveness determined individually for each of the plurality of partitions.

4. The method of claim 1, wherein the rough filtered partitions corresponding to one of the plurality of partitions is discontinuous from the rough filtered partitions corresponding to a neighboring partition of the plurality of partitions.

5. The method of claim 1, further comprising:
mirroring, by the processor, a portion of the raw analyte data corresponding to at least one of a beginning of the raw analyte data or an end of the raw analyte data to form mirrored data at the beginning or end of the raw analyte data for generation of the smooth filtered data; and
removing, by the processor, a portion of the smooth filtered data corresponding to the mirrored data.

6. The method of claim 1, wherein at least one of the plurality of partitions overlaps another of the plurality of partitions.

7. The method of claim 1, wherein the smoothing algorithm is configured to smooth over missing data in the raw analyte data to generate the smooth filtered data as continuous through the missing data.

8. A system for noise reduction in analyte data, the system comprising:
an analyte sensor system configured to generate raw analyte data for a user;
a memory comprising executable instructions;
a processer in data communication with the memory and configured to execute the instructions to:
receive the raw analyte data corresponding to a noisy signal trace from the analyte sensor system;
partition the raw analyte data into a plurality of partitions;
apply an adaptive filter separately to each partition to generate rough filtered partitions with a reduced noise component in each partition;
apply a smoothing algorithm across the partitions to smooth the rough filtered partitions from each partition to generate smooth filtered data forming a single smoothed signal trace; and
display the smooth filtered data to the user.

9. The system of claim 8, wherein at least one of the plurality of partitions overlaps another of the plurality of partitions.

10. The system of claim 8, wherein the adaptive filter is configured to implement a filtering aggressiveness determined individually for each of the plurality of partitions.

11. The system of claim 8, wherein the rough filtered partitions corresponding to one of the plurality of partitions is discontinuous from the rough filtered partitions corresponding to a neighboring partition of the plurality of partitions.

12. The system of claim 8, wherein the processor is further configured to:
mirror a portion of the raw analyte data corresponding to at least one of a beginning of the raw analyte data or an end of the raw analyte data to form mirrored data at the beginning or end of the raw analyte data for generation of the smooth filtered data; and
remove a portion of the smooth filtered data corresponding to the mirrored data.

13. The system of claim 8, further comprising determining a noise variance profile for the raw analyte data based on a noise component determined for each of the plurality of partitions.

14. The system of claim 8, wherein the smoothing algorithm is configured to smooth over missing data in the raw analyte data to form the smooth filtered data to be continuous.

15. A computer-readable medium comprising instructions which, when executed by a processor, cause the processor to perform a method for noise reduction in analyte data, the method comprising:
receiving raw analyte data corresponding to a noisy signal trace from an analyte sensor;
partitioning the raw analyte data into a plurality of partitions;
applying an adaptive filter separately to each partition to generate rough filtered partitions with a reduced noise component in each partition; and
applying a smoothing algorithm across the partitions to smooth the rough filtered partitions from each partition to generate smooth filtered data forming a single smoothed signal trace.

16. The computer-readable medium of claim 15, wherein at least one of the plurality of partitions overlaps another of the plurality of partitions.

17. The computer-readable medium of claim 15, wherein the adaptive filter is configured to implement a filtering aggressiveness determined individually for each of the plurality of partitions.

18. The computer-readable medium of claim 15, wherein the rough filtered partitions corresponding to one of the plurality of partitions is discontinuous from the rough filtered partitions corresponding to a neighboring partition of the plurality of partitions.

19. The computer-readable medium of claim 15, wherein the method further comprises:
mirroring a portion of the raw analyte data corresponding to at least one of a beginning of the raw analyte data or an end of the raw analyte data to form mirrored data at the beginning or end of the raw analyte data for generation of the smooth filtered data; and
removing a portion of the smooth filtered data corresponding to the mirrored data.

20. The computer-readable medium of claim 15, wherein the method further comprises determining a noise variance profile for the raw analyte data based on a noise component determined for each of the plurality of partitions.
